(19) United States
(12) Patent Application Publication
(10) Pub. No.: US 2017/0164886 A1
(43) Pub. Date: Jun. 15, 2017

(54) Nanostructured Biomimetic Devices of Making and Its Therapeutic Applications Thereto

(71) Applicant: ELLEN T. Chen, ROCKVILLE, MD (US)

(72) Inventor: ELLEN T. Chen, ROCKVILLE, MD (US)

(21) Appl. No.: 15/352,658

(22) Filed: Nov. 16, 2016

Related U.S. Application Data
(63) Continuation-in-part of application No. 13/919,216, filed on Jun. 17, 2013, now Pat. No. 9,534,999.

(51) Int. Cl. A61B 5/00; A61B 5/053; G01N 27/327; A61B 5/01; G01N 33/574; G01N 33/487

(52) U.S. Cl. CPC A61B 5/4312; G01N 33/574; G01N 33/48792; G01N 33/48735; G01N 33/57415; G01N 27/3276; A61B 5/015; A61B 5/053; A61B 5/4848; A61B 5/4866; A61B 2560/0431; A61B 2562/0214; A61B 2562/0285

(57) ABSTRACT

The present invention provides a handheld electrochemical cancer monitoring and therapeutic device comprising a key component of a sensor compartment comprising an electrode with a substrate having a nanostructured biomimetic membrane attached thereto and an open window allows the electrode fixed in a socket of the sensor compartment to the window to be contacted to the human breast; in the base of the device there is a window for cancer heat release result displayed in a contour map format when the device applied a fixed current to induce cancer cell's bio-communication with the sensor membrane selectively under noninvasive, antibody-free and reagent-free conditions. Methods for therapeutic function and monitoring of early asymptomatic normal human breast in vivo and therapeutic treatment of breast cancer cell line in vitro are disclosed.

### CROSS REFERENCE TO RELATED APPLICATIONS

This patent application entitled “Nanostructured Biomimetic Devices of Making and Its Therapeutic Applications Thereto” is a continuation in part non-provisional patent application that claims the benefit of U.S. Non Provisional patent application Ser. No. 13/919,216 entitled “A Contour Map of Multiple Variable Correlation Method Assesses the Heat Released by Cancer Cells and Monitoring the Cancer Progress Using Nanostructured Biomimetic Devices” filed on Jun. 17, 2013 and claims the benefit of U.S. provisional application No. 61/660,072, filed 15 Jun. 2012 and entitled “Nanopore Structured Biomimetic Sensor Device;” is a nonprovisional of U.S. provisional application No. 61/660,080, filed 15 Jun. 2012 and entitled “Nanopore Structured Biomimetic Sensor Device”; is a nonprovisional of U.S. provisional application No. 61/660,690, filed 16 Jun. 2012 and entitled “Nanopore Biomimetic Device;” and is a nonprovisional of U.S. provisional application No. 61/691,632, filed 21 Aug. 2012 and entitled “Nanopore Structured Biomimetic Device.” The disclosure of each aforementioned application is incorporated herein by reference in its entirety.

### FIELD OF THE INVENTION

The present invention relates to the field of cancer monitoring and, in particular, to handheld devices comprising a nanostructured biomimetic membrane, through functions of spontaneously discharging electric pulses and charging electric pulses at real time, hence the heat released by cancer cells can be mapped in the contour form and the degree of the cancer prognosis severity can be monitored.

### BACKGROUND OF THE INVENTION

It is a well recognized phenomenon that cancer cells have abnormal cell membrane potential [1-3]. The conventional bio-potential method used for diagnosing cancer lacks of sensitivity and selectivity [3]. Biologists measure cell membrane action and resting potentials with burdensome instrumentation with time consuming procedures. A recent report shows breast cancer cell division caused a membrane potential increase [4] due to variations in ion channel expression. However. the method requires a time consuming large computer algorithm for modeling, and still lacks selectivity and sensitivity. A recent paper reported that the measured neural cell membrane spiking potential has a signal to noise ratio of 2 [5]. Because the normal cell membrane action potential is 58 mV, and −70 mV is for the resting potential [6], the small signals are very easily buried in the background noises [7] that can cause problems to pediatric neurologist and intensive care unit doctors who need strong signals to monitor and diagnose the neonatal neurological diseases [7]. There is very few, if any, to build a device that can induce receptors of cancer cells spontaneous and direct interact with the artificial receptor of the membrane of the device without using antibody or labeling. The amplified signals are several orders of magnitude higher in signal to noise ratio than the conventional methods, will provide means to enhance the sensitivity and selectivity of the detection. The goal of this invention is to develop such a handhold device by fabricating a nanopore structured biomimetic membrane on a gold chip with an imidazolium receptor in the polymer network to induce the direct biocommunication to cancer surface receptors without using antibody, and without labeling in order to overcome the current technology drawbacks and through the functions of discharge and change electric pulses at real time, hence the transformed energy from the cancer cells can be mapped out in three dimension with a contour form, therefore the progress of the cancer can be monitored visually, that will be beneficial to patients and medical doctors.

### SUMMARY OF THE INVENTION

The present invention provides a novel electrochemical device and a cancer cell heat release map method to visually display the cancer progresses. The novel device comprises an electrode having a nanopore biomimetic electron-relay network with imidazolium-ATP of cancer cell-water-pyridine at the active sites that mimic the electron-relaying between His 516 and N(5)-FAD of GOx for the purpose to selectively detect triple-negative breast cancer cell at single cell concentration and it rejects brain cancer cell. The device is fast in millisecond to detect cancers without sample preparation and without interference from other substances, such glucose and proteins under reagent-free conditions. A unique biomarker of the ratio of “Action/Resting” cell membrane potential can be used to monitor the cancer progress against the normal cells. A visual contour map of a multiple variable correlation method provided to assess the heat release from the cancer cells against the normal cells is presented. The device for a potential therapeutic application was demonstrated by discharge voltage pulses from the live cancer cells with release extra energy that the cells possessed until it returns to a normal status in terms of normal cell membrane action/resting potential ratio.

is an object of the present invention to provide a new generation of cancer detection device has selectivity among single cancer cell between brain cancer and breast cancers through a nanostructured biomimetic membrane sensing device under antibody-free an labeling-free conditions. It is an object of the present invention to provide above described device system with another function of visualizing 2D contour heat release map from the cancer cells based on a multiple variable energy conversion method.

It is another object of the present invention to establish a ratio of cell membrane action potential vs. resting potential as the land marker ratio of “action potential” vs. “resting potential” (RAPRP) values that distinguish between the normal cells and the cancer cells, as the biomarker, under antibody-free and labeling-free conditions. It is a further object of the present invention to provide a device having therapeutic function of healing of breast cancers by inducing the cancer cells to release extra energy that it possessed through spontaneously discharge electric pulses, while real time monitoring the ratio of action/resting membrane potential/ heat release map.

### Following are the Summary of Invention for the CIP Application

It is an object of the present invention to provide a new generation of therapeutic electrochemical energy devices for in vitro healing of low abundant cancer cells by inducing the cancer cells to spontaneous release extra energy that it possessed through discharge electric pulses and the pulses are not harmful to the normal tissue or normal cell under antibody-free an labeling-free conditions.

It is an object of the present invention to provide a new generation of therapeutic electrochemical energy device offering no harm to normal healthy breast tissue in vivo under noninvasive and pain-free condition at real time.

It is an object of the present invention to provide a new generation of therapeutic electrochemical energy device offering no harm to normal healthy breast tissue in vivo with appropriate current dosages.

It is an object of the present invention to provide a visualized heat release map method to monitor the cancer progress.

It is an object of the present invention to provide a new generation of therapeutic electrochemical energy device with all components assembling to be a system that can be portable, for in vitro and in vivo use with real time heat release display functions and the heat can be quantifiable with high sensitivity and precision.

### BRIEF DESCRIPTION OF THE DRAWINGS

FIG. 1 Illustrates an art work for the model used to construct the Au-nanopored His 516 receptor-CD SAM electrode. The moiety of the receptor-CD was cross-linked with PEG and PVP and a nanopore structured SAM was self-assembled.

FIG. 2 (A) illustrates the initial step of forming the electron-relaying system in an imidazolium-water-pyridine before glucose entered the nanopore. (B) illustrates the second step of “host-guest” inclusion between imidazolium and glucose when glucose solution was added in the buffer. (C) illustrates the third step of electron-relay system. It has rearranged to form an imidazolium-glucose-water-pyridine electron-relaying system at the active sites that mimic the electron-relaying between His 516 and N(5)-FAD of GOₓ through the hydrogen bonding.

FIG. 3A shows two-dimensional atomic force microscopy (AFM) image of nanopore structured mM-β-DMCD/PEG/PVP, here mM-β-DMCD's short name as MCD, SAM with an internal receptor imidazolium. Brighter areas represent higher topography. The roughness measurements Peak-to-Valley (Z range), Root Mean Square (RMS), and Average Roughness (Rₐ) are also shown for this image.

FIG. 3B shows 3D AFM image for the same sensor as in FIG. 3A.

FIG. 3C shows the cross-section analysis of nanopore size measurement of the same sensor as in FIG. 3A.

FIG. 4A shows an image of the human breast cancer cells of MDA-MB-231 in a growing medium of DMEM.

FIG. 4B shows an image of the human brain cancer cells of Glioblastoma brain cancer line SNB-19 in a growing medium of DMEM.

FIG. 5A illustrates the effect of current on Double Step Chronopotentiometry (DSCPO) profiles without incubation and without inhibitor under 5 cancer cells/mL concentration with current change from a to d: 50 nA, 50 μA, 10 mA, 30 mA; e: 50 pA, f: control without cell;

FIG. 5B illustrates the 5 breast cancer cells heat released without incubation.

FIG. 6A Illustrates the effect of current change on DSCPO profiles with 1 day incubation and without inhibitor under 5 cancer cells/mL concentration with current change from 10 mA (a), 50 μA (b), 50 μA (c) and without cancer cell (d).

FIG. 6B illustrates the 5 breast cancer cells heat released with 24 hrs incubation

FIG. 7A illustrates current change effects on the DSCPO profiles of a normal living breast cell in a non invasive manner from a to e: 50 pA (a), 50 nA (b), 50 μA (c), 30 mA (d) and 20 mA (e), respectively.

FIG. 7B illustrates the normal breast cell heat release map.

FIG. 8 illustrates the ameprometric profiles using the AU/MCD sensor. breast cancer cell (1 cell/mL) with incubation without inhibitor; (B) Breast cancer cell (1 cell/mL) with incubation and with an inhibitor; (C) Brain cancer cell (1 cell/mL) with incubation without inhibitor; (D) Brain cancer cell (1 cell/mL) without incubation and. without inhibitor in cell culture media,

FIG. 9A illustrates the breast cancer detection device selectively detecting live single breast cancer cell (1 cell/mL) over live brain cancer cell (1 cell/mL) after 24 hrs incubation against control without cancer cell.

FIG. 9B depicts the cancer cell's bio-communication in an electron-relay between the ATP of the cancer cell and the imidazolium, water, pyridine groups at the active sites that mimics the electron-relay network between His 516 and N(5)-FAD of GOₓ in the presence of glucose.

FIG. 10 illustrates CV profiles of pulse effects on live breast cancer cell signal with Au/MCD sensor at 50 cell/mL in NIST standard human serum SRM 965A with certified level 1 glucose at room temperature: A: cancer cell only; B: After the first pulse at −10 μA, 50 s and the second pulse at −100 μA, 50 s; C: same as B, plus a rest for 3 minutes; D: After C, then discharged the third pulse at −100 μA, 50 s; E: Control.

FIG. 11 illustrates the therapeutic treatment profiles using an Au/MCD/PEG/PVP sensor on breast cancer 50 cell/mL in NIST standard human serum SRM 965A with blood glucose level 1 discharges at pulses (a): control at −10 mA, 50 s; (b): control at −100 mA, 50 s; (c): cancer cell at −10 mA, 50 s; (d): cancer cells discharge the second pulse at −100 mA, 50 s; (e), cancer cell discharges after the third pulse treatment at −100 mA, 50 s at room temperature. The pulse treatment method was used the single step chronopotentiometry method (SSCPO).

FIG. 12 illustrates three replicates of DSCPO curves of breast cancer 50 cell/mL in NIST standard human serum SRM 965A with certified blood glucose level 1 tested on Au/MCD/PEG/PVP sensor at room temperature at ±10 mA without any pulse treatments.

FIG. 13 illustrates the evaluation of the efficacy of the pulse treatment based on FIG. 11 on breast cancer cells 50 cell/mL, in MST reference human serum SRM 965A with certified blood glucose level 1 tested at ±10 mA, each step 0.004 s: (a) depicts 50 cancer cell/mL without pulse treatment; (b) After 2 pulse; (c) After the third pulse treatment.

FIG. 14A depicts the corresponding in vitro heat release map according to FIG. 13 curve a. FIG. 14B depicts the corresponding in vitro heat release map as shown in FIG. 13 curve b. FIG. 14C depicts the corresponding in vitro heat release map as shown in FIG. 13 curve c.

FIG. 15 depicts the monitoring processing of the cancer heat release map evaluated the efficacy of the pulse treatment based on FIG. 11 on breast cancer cells 50 cell/mL in NIST reference human serum SRM 965A with certified blood glucose level 1, and the CV method evaluation curves were shown in FIG. 10, (a) with 50 cancer cell/mL without pulse treatment; (b) After 2 pulse and rested for 3 minutes; (c) After the third pulse treatment.

FIG. 16A depicts the profile curves of the three pulses at first pulse −10 μA, second pulse −100 μA, and third pulse −100 μA treatment with each pulse duration of 50 s with data rate 50 kHz for a normal healthy female subject's normal breast by the SSCPO method. FIG. 16B depicts the profile SSCPO curves of the three pulses in same conditions, except each pulse at −5 mA.

FIG. 17A depicts the evaluation of energy change using a DSCPO method at the therapeutic dosages as shown in FIG. 16 A of −10 μA, −100 μA compared with the control. Each sample runs duplicate or triplicates. FIG. 17B depicts the evaluation of energy change as shown in FIG. 16B compared with the controls.

FIG. 18 depicts the heat contour map for monitoring the before and after pulse treatment of the energy change as shown in FIG. 17A, a control female subject with the ratios of action/resting potential as they axis with the values kept around 0.9-1.1 normal range during three pulses treatments compared with before treatment, the x axis values are the total time elapse during the entire treatment, and the z in heat release calorie/kg. The kg refers to the weight of the sensor. The red labels are for the locations of the pulses.

FIG. 19 depicts the scheme of the assemble of the cancer monitoring system consists of “1”, a flexible sensor which is contacted with a cleansed area near the areola of a breast; “2” refers to a breast, and a transmitter with embedded software to transmit the signal wirelessly to a receiver with a receiver/displayer for 3D mapping of heat release in either by ratio of action/resting potential (as y axis), log current as x axis and heat release calorie as Z axis); “3” or by a progress mapping of ratio of action/resting potential as y axis, total pulse therapy time as x axis and heat release calorie as z axis shown in “4”.

FIG. 20 depicts the handheld prototype device components for cancer detection, monitoring and therapeutic function under in vivo, pain-free and non-invasive conditions. The top figure is a bird-eye view of the device system is presented. “1” represents the base of the device. “2” represents the slid for depositing sensor. The left side bottom figure is a face-to-face view of the device. “3” refers to the contact surface. “4” is the display window for the color coded contour heat release map; the middle figure is the side-view of the device. “5” is an activation button. The far right side figure is a back-view of the device with “6” is a USB and wireless interfaces.

FIG. 21A depicts the face-to-face view of the device. FIG. 21B depicts the battery charging contacts. FIG. 21C is a close view of the device with the cancer heat releasing map on the display at the bottom.

FIG. 22 depicts the components in the top head of the device. “100” is an open window exposed the sensor allowing it direct contacts with the breast, and the sensor was inserted inside the slot of the sensor compartment of the device. The open window and the slot are perpendicular to each other. Inside of the slot has a socket for fixing the sensor in the open window. “101” is the device contact surface. “102” is the sensor. “103” is the A/D converter and the embedded software chip for transmission and converting signal and an interface card.

### DETAILED DESCRIPTION OF THE INVENTION

### EXAMPLE 1

### Fabrication of the Nanostructured Biomimetic Self-Assembling Membranes (SAM)

Reagent grade poly (4-vinylpyridine) (PVP), polyethylene glycol diglycidyl ether (PEG), were purchased from Aldrich-Sigma. The PVP was recrystallized in methanol. The mono imidazol derivative dimethyl β-cyclodextrin (mM-β-DMCD) was generally synthesized according to the published procedures [8]. The gold chips were purchased (Fisher Scientific) and the mixture solutions with proper compositions and procedures were followed by published literature in [9].

### EXAMPLE 2

### Characterization of the Membrane of AU/SAM

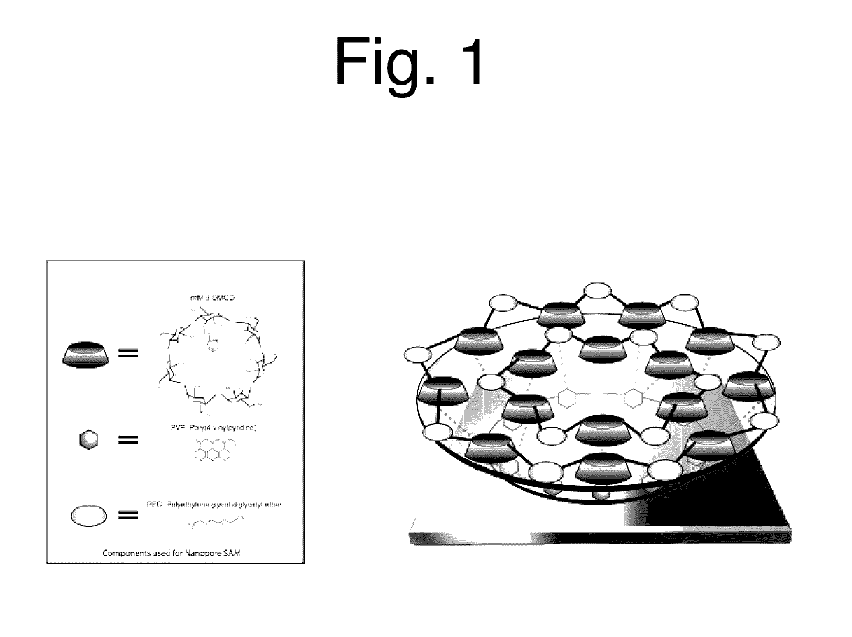

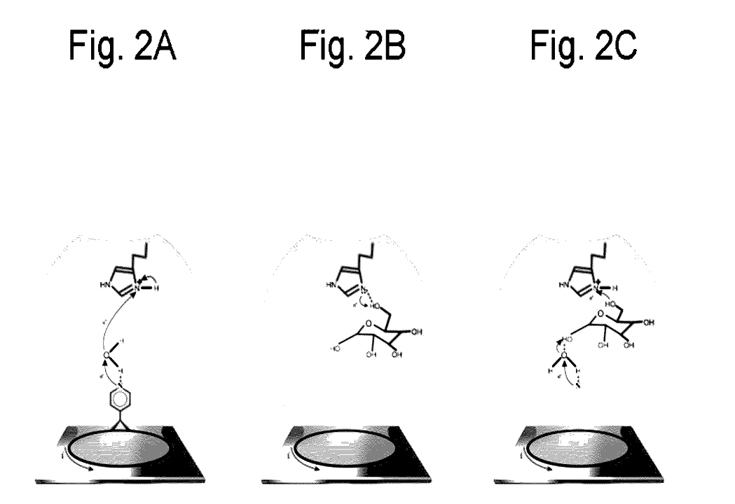

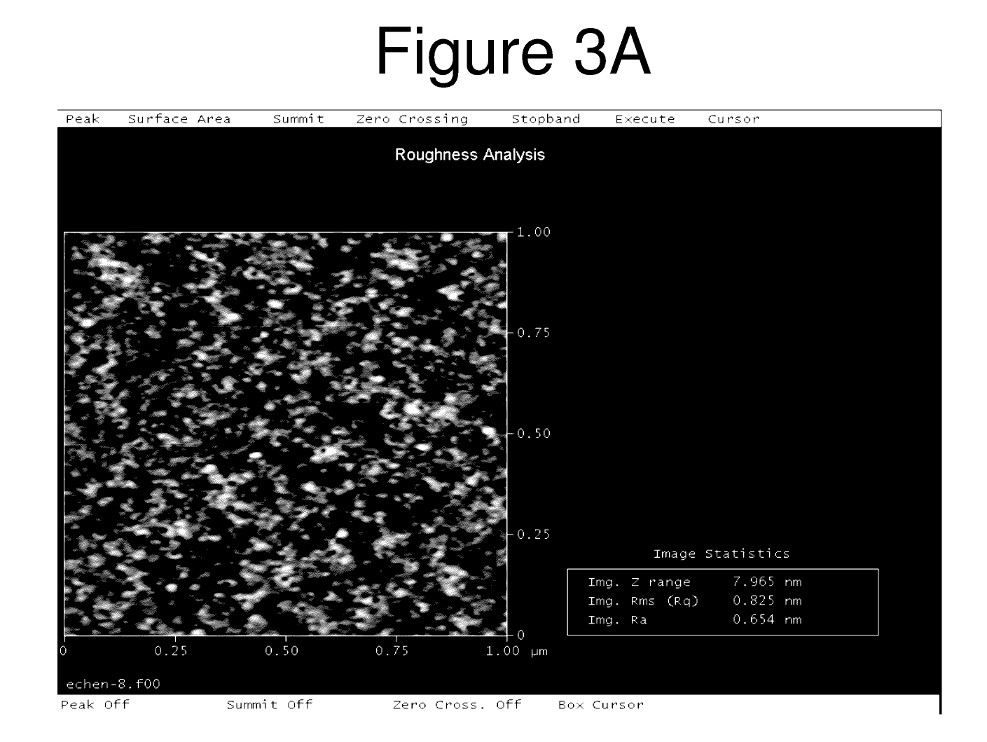

The morphology of the AU/SAM was characterized using a Dimension 3100 Atomic Force Microscope (AFM) (Bruker Nano, CA.). FIG. 1 is an art illustration of the model used to construct the Au-nanopored sensor cross linked with polymers and modified cyclodextrins by SAM method. FIG. 1 illustrates an art work for the model used to construct the Au-nanopored His 516 receptor-CD SAM electrode. The moiety of the receptor-CD was cross linked with polyethylene glycol diglycidyl ether (PEG) and poly(4-vinylpyridine) (PVP) and self-assembled a nanopore structured SAM through hydrogen bonding. Possible driving forces to form such a nanopore could be the changes in the heat of formation in the active site and the change of free energies of solvation that are favorable to the electron-relay processing. The pKₐ value difference between the receptor His 516 and the pyridine, and the difference of hydrophobicity between the internal cavity of CD and the pyridine of PVP may also play an important role in the self-forming nanopore as shown in FIG. 2. FIG. 2 illustrates the proposed electron-relaying model in the active sites with and without glucose entered the nanopore. FIG. 2(A) illustrates the initial step of forming the electron-relaying system of the imidazolium-water-nitrogen relay before glucose entering the nanopore. Observations of the DET current confirmed the electron transfer step using a cyclic voltammetric scan method. FIG. 2(B) illustrates the second step of “host-guest” inclusion between imidazole and glucose when adding glucose solution in the pH 7 buffer. This step temporally disturbed the electron-relay flow, that indicates the analyte glucose entered the nanopore and had an influence on the DET current flow. FIG. 2(C) illustrates the third step of electron-relay system that has rearranged to form an imidazolium-glucose-water-pyridine electron-relaying at the active sites that mimic electron-relaying between His 516 and N(5)-flavin adenine dinucleotide (FAD) of GOₓ.

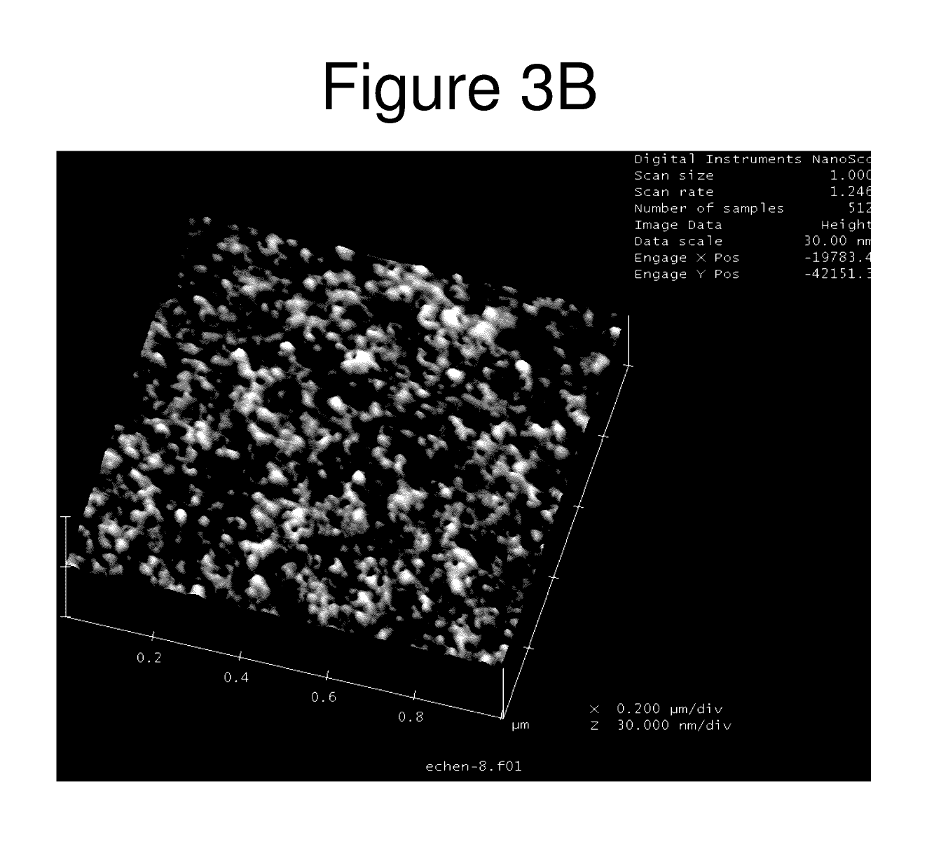

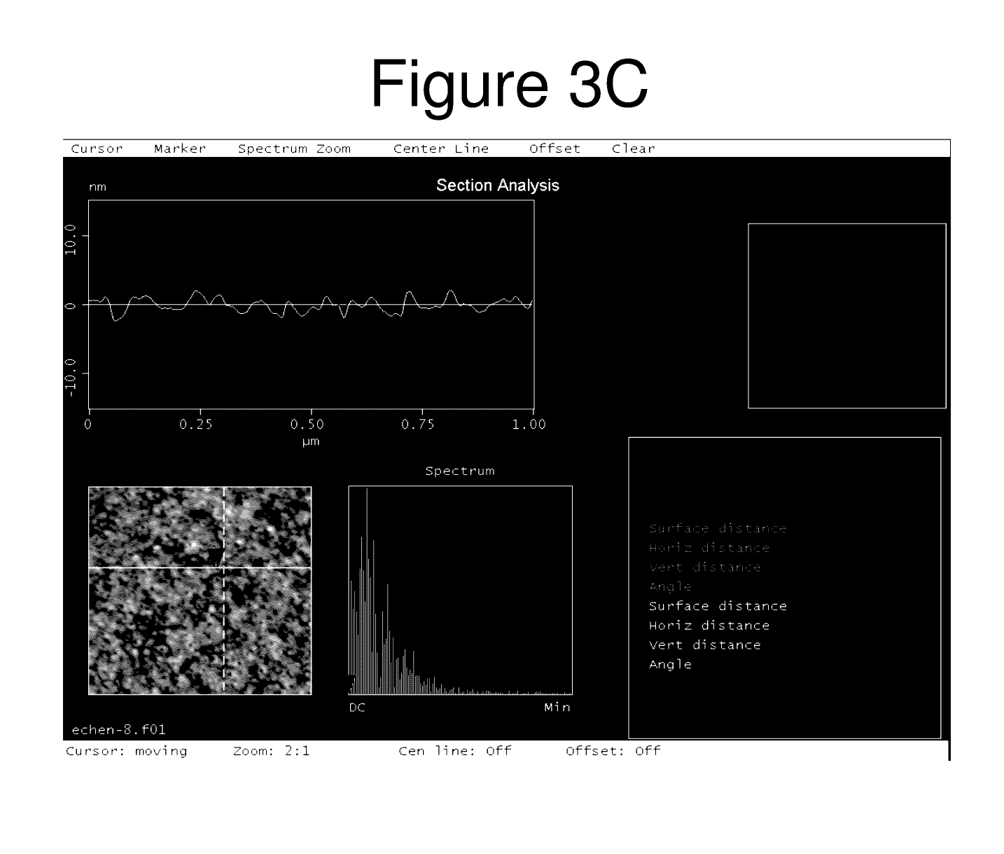

The surface structure, shown in FIG. 3A, was scanned by TappingMode AFM using a silicon cantilever and a tip with a 5-10 nm radius and resonance frequency of 300 kHz [10]. The roughness of the SAM was 0.82 nm Root-Mean-Square (RMS) as shown in FIG. 3A and 3B. FIG. 3C is a cross-section analysis with pore size measurement as 19.5 nm in average.

### EXAMPLE 3

### Human Cancer Cell Line MDA-MB-231 and the Glioblastoma Brain Cancer Line SNB-19

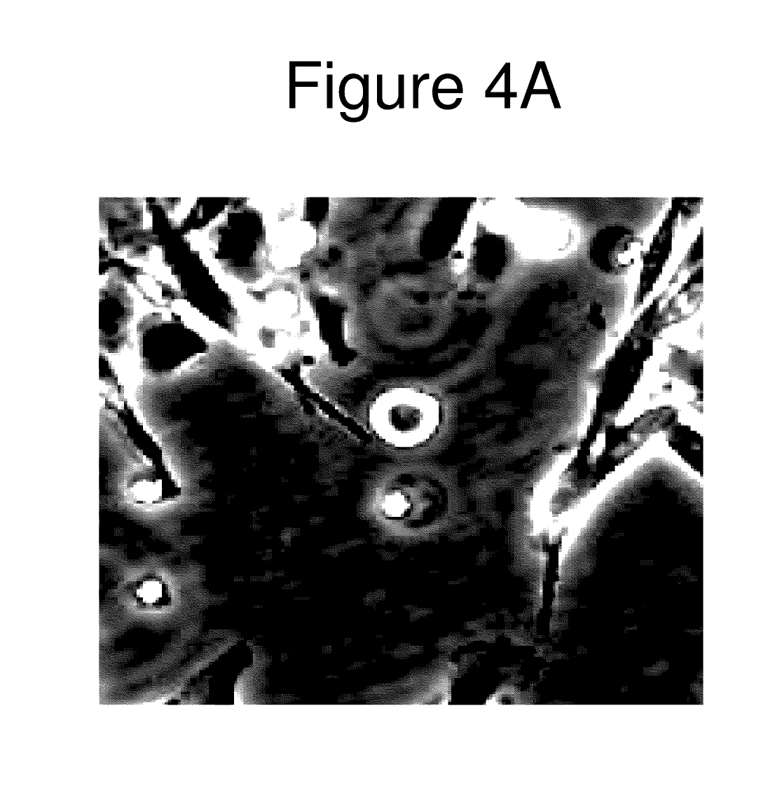

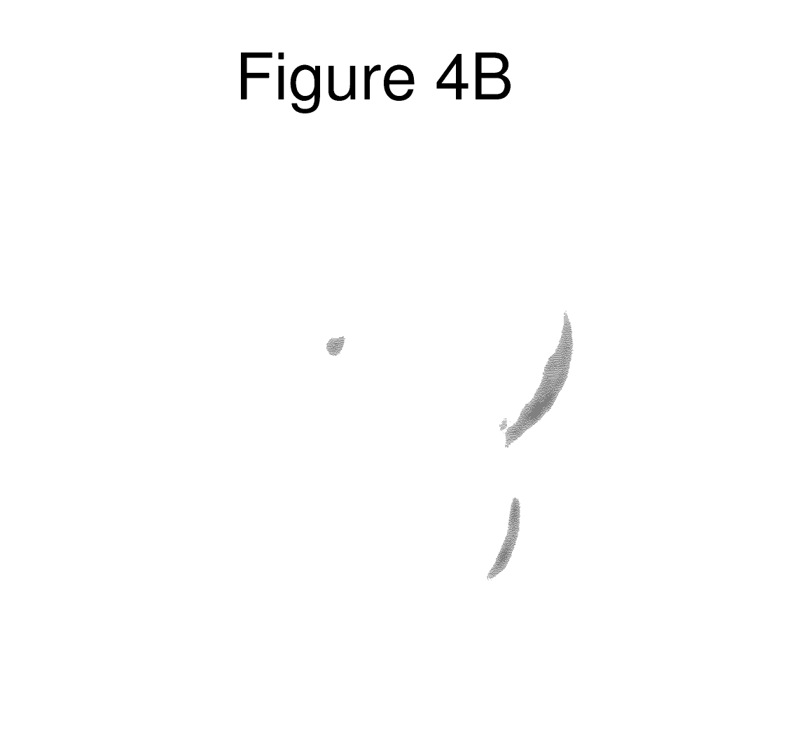

Breast cancer cell samples are human adenocarcinoma cells line MDA-MB-231 as shown in FIG. 4A taken from breast cancer tissue. The glioblastoma brain cancer cells samples are human neuroblastoma line SNB-19 as shown in FIG. 4B. The cell cultures are held in a base growing medium of DMEM (Dulbecco/Vogt Modified Eagle's minimal essential Medium—a common growth culture medium used for human cell incubation) (Invitrogen, CA infused with a 10% concentration of FBS (fetal bovine serum), 10 mM HEPES, 100 units/mL penicillin/Streptomycin and 2 mM L-glutamine. It was kept in a normal atmosphere at a temperature of 37.0° C. with 10% CO₂ and humidified air. The cancer cells in the DMEM media were incubated for 24 hrs. Before test the cancer cells, dilution procedures were conducted.

### EXAMPLE 4

### The Single Cancer Cell Selectivity

The selectivity of the sensor device towards detecting the breast cancer cells compared with that of normal living breast cells were conducted at room temperature by the Double Step Chronopotentiometry (DSCPO) method. The normal breast specimen was tested by the DSCPO method in a non-invasive manner, that the wetted sensor was directly attached on the skin of the breast of the subject, whom was consent and was approved with the IRB.

The DSCPO method was used for evaluation of the sensor performance for cancer detection under fixed current conditions. Changes of current effects on the “action potential” and “resting potential” were conducted in the range from pA to mA in vitro culture medium at room temperature. All experiments were finished within 1 hr. Changes of cell concentrations effect on the potentials were conducted in the ranges from 1, 5, 100, to 200 cell/mL using an electrochemical work station (Epsilon, BASi, IN). The 16 channel AU/SAM electrode chip configuration was mentioned in Section of Fabrication of the Nanostructure Self-Assembling Membrane (SAM) Gold Sensor Chip. The center circular electrode is the working electrode, and the adjacent gold electrodes are the auxiliary and the reference electrode, respectively.

Selectivity was further confirmed by an amperometric method using the MCD sensor as shown in FIG. 8. It demonstrates this sensor selectively detecting breast cancer cell over brain cancer cell at 1 cell/mL concentration. FIG. 8 illustrates the ameprometric profiles. (A) refers to the breast cancer cell with incubation without inhibitor; (B) refers to breast cancer cell with incubation with an inhibitor; (C) refers to the brain cancer cell with incubation without inhibitor; (D) refers to the brain cancer cell without incubation without inhibitor. It was observed that the inhibitor reduced the breast cancer signal strength by 92%.

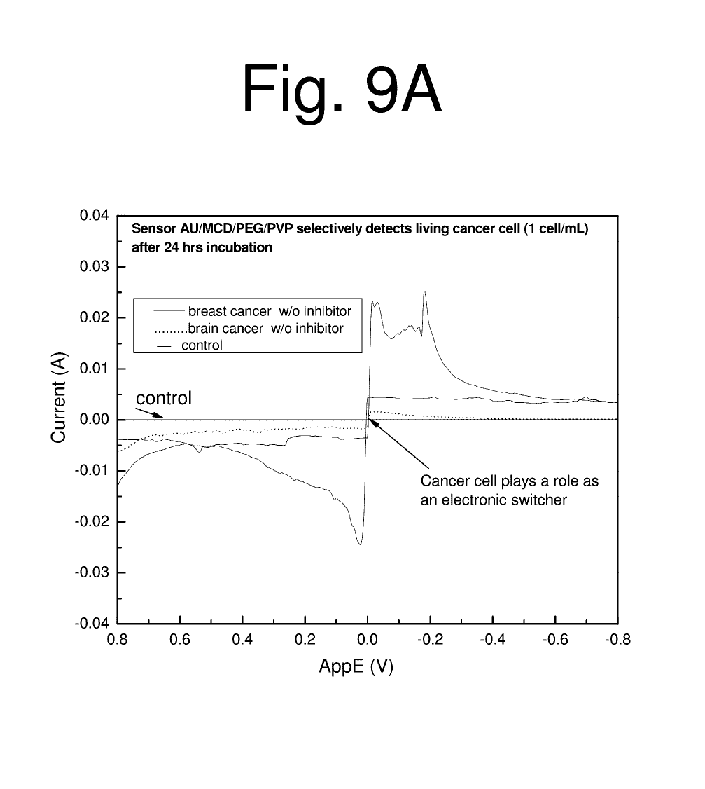

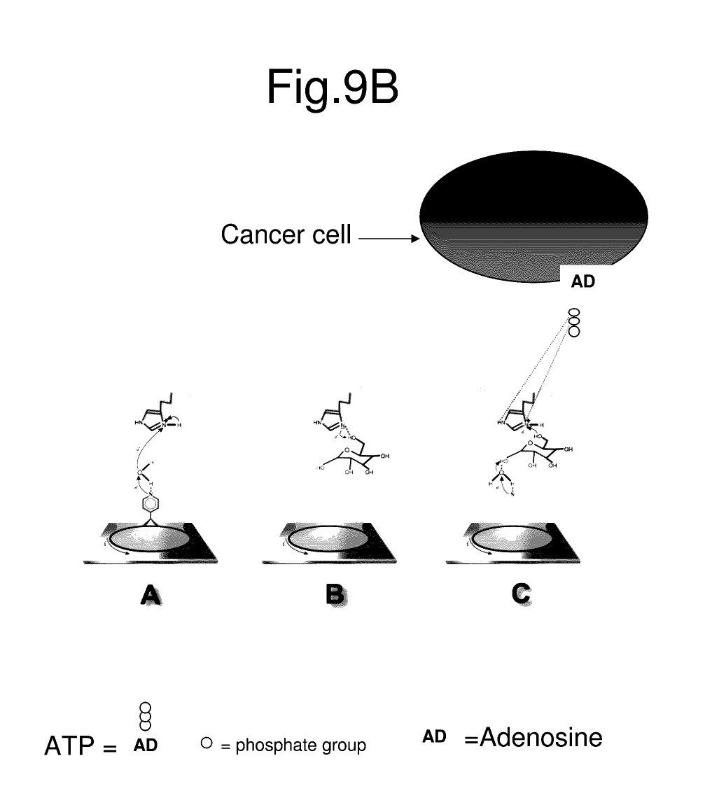

The selectivity study was also conducted at room temperature by a Cyclic Voltammetric method (CV) to detect the breast cancer cells and the brain cancer cells. The scan rate was constant at 20 mV/s for the CV method. It was shown in FIG. 9A, that the MCD sensor only selectively detects breast cancer cell with peaks signal intensities of ±0.025 A at 0.0 mV for the solid red curve over brain cancer cell without peak observed of (the black dotted line) at 1 cell/mL concentration against control in blue solid line.

### EXAMPLE 5

### Effect of Current Change with or Without Cell Incubation

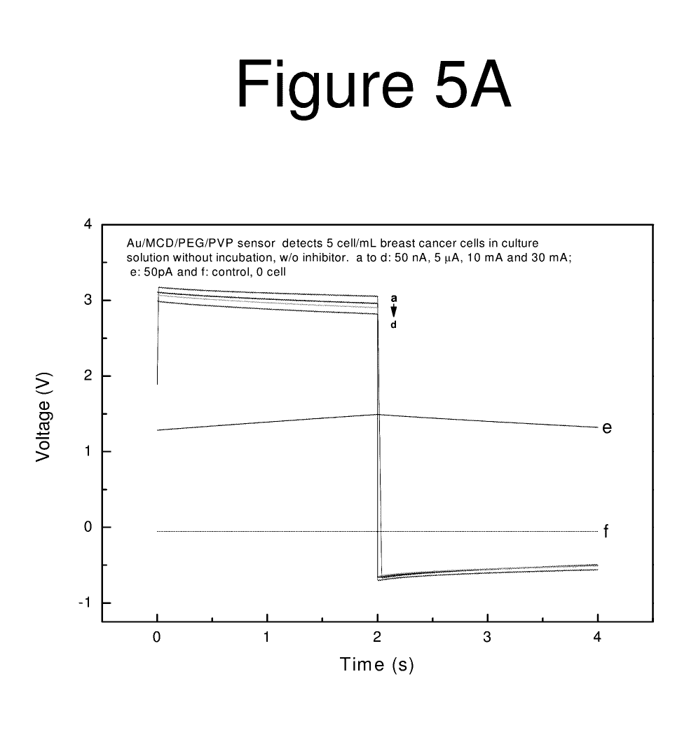

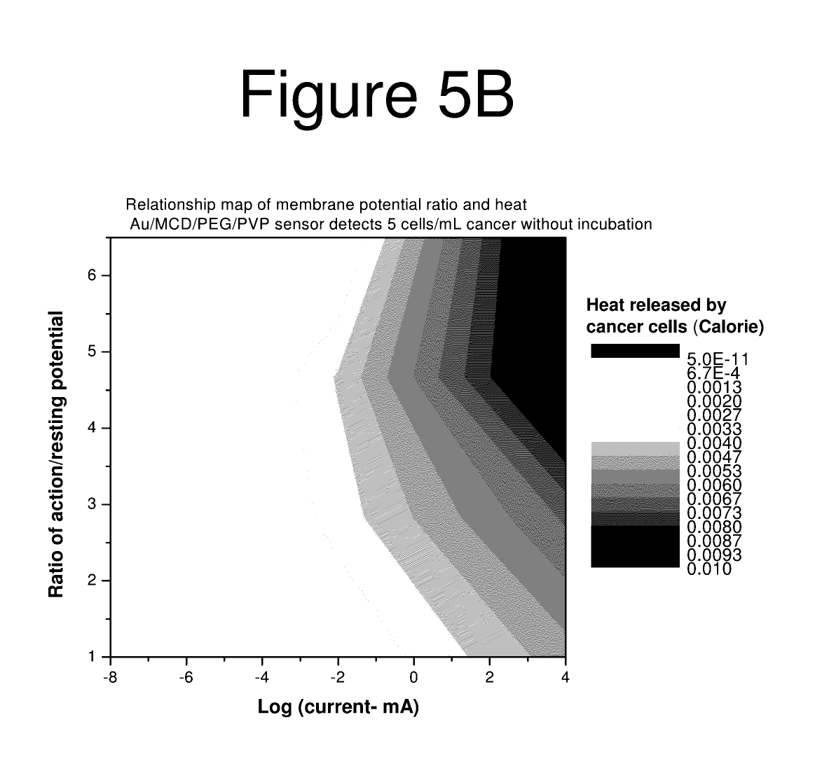

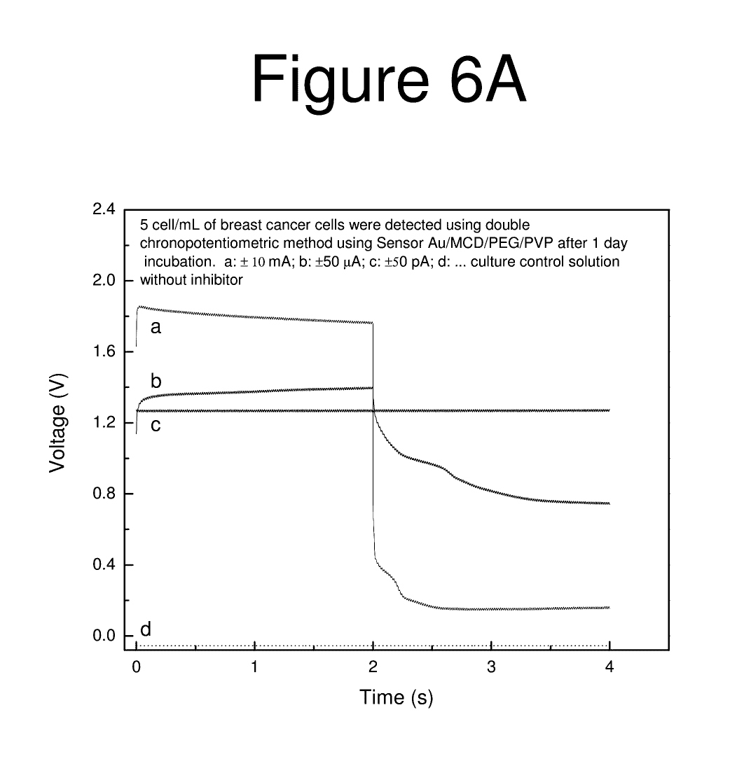

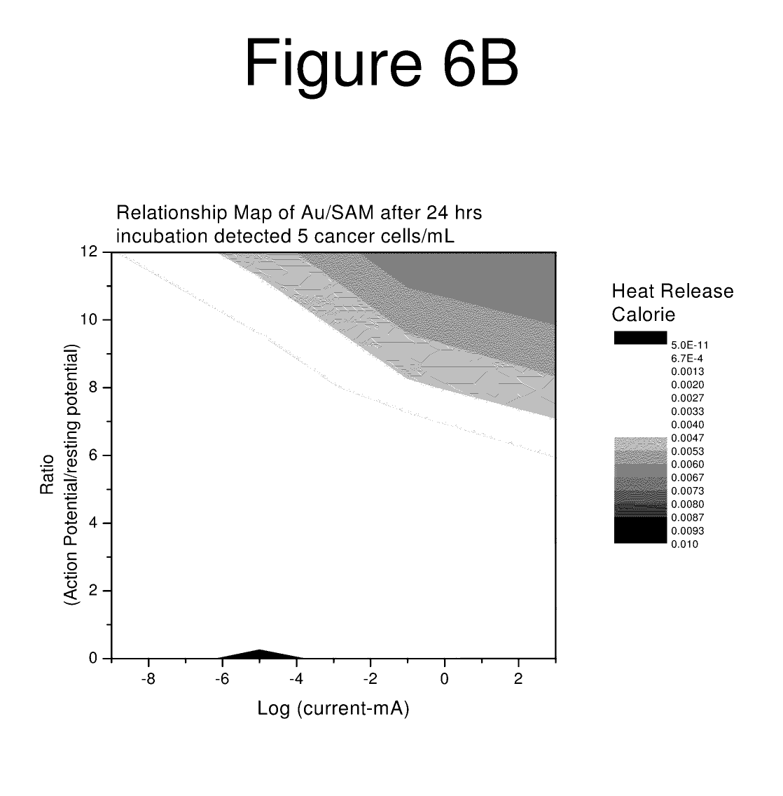

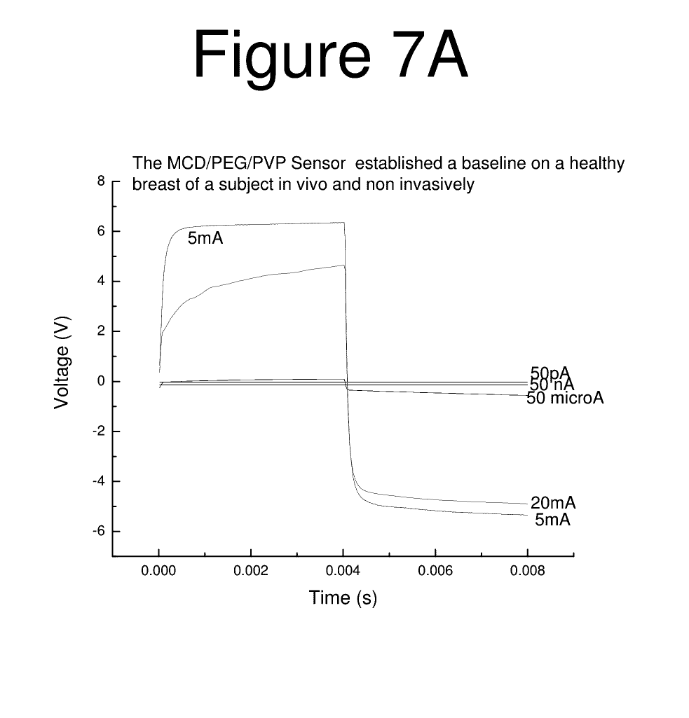

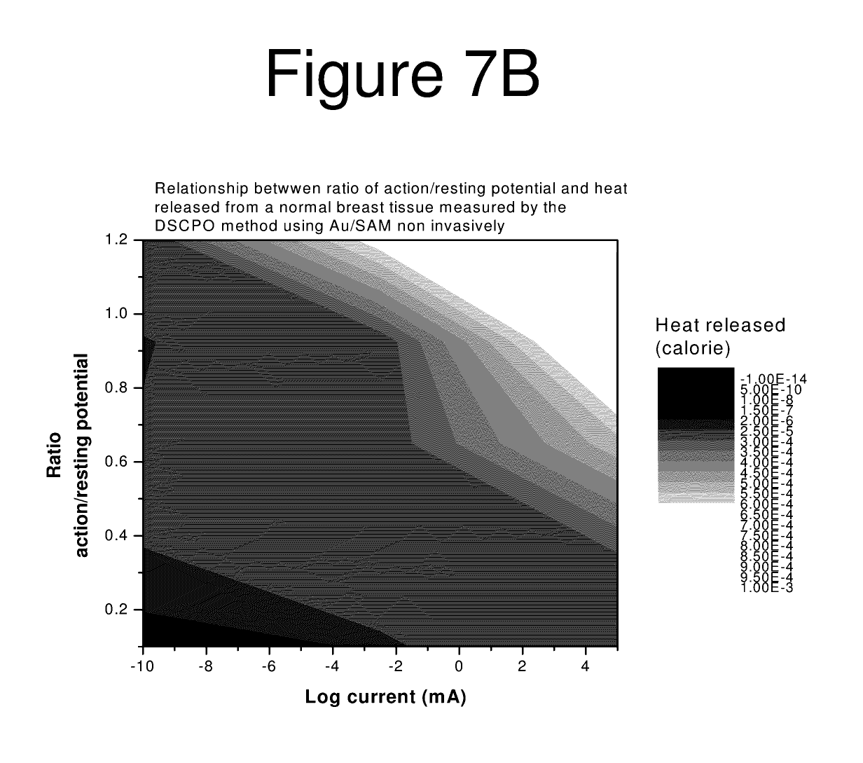

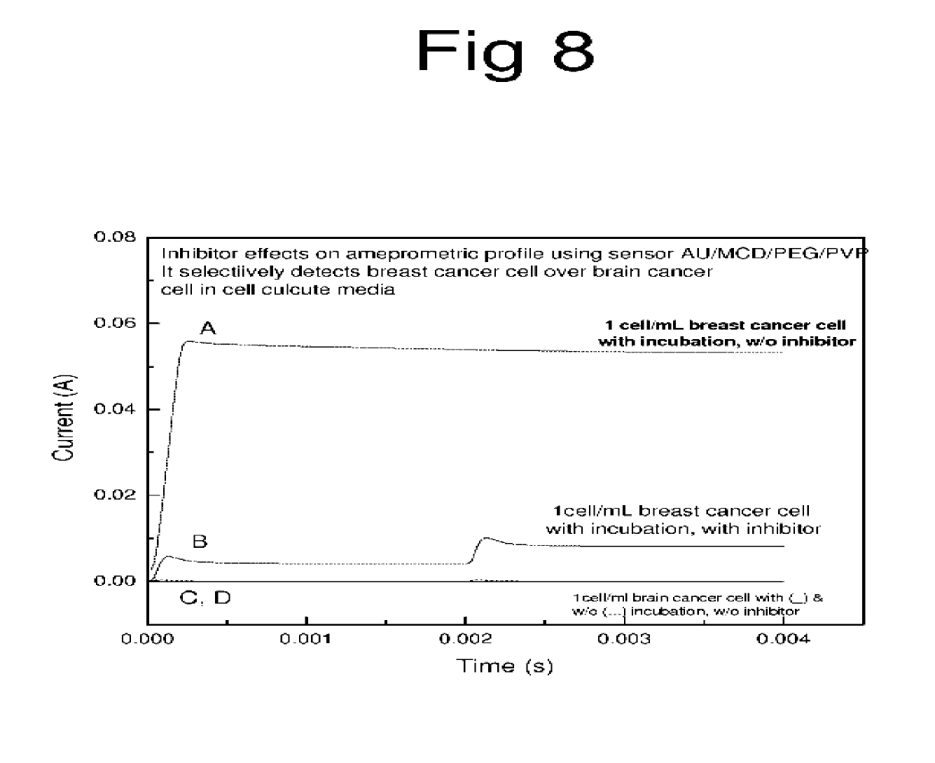

FIGS. 5A and 6A illustrate current change effect on the DSCPO profiles under 5 breast cancer cell/mL concentration with or without 1 day incubation, respectively, against the controls that did not have cancer cell as shown in FIG. 7A. Both figures were without inhibitors. It is obvious that with 1 day incubation, the DSCPO's action and resting potential profiles moved up to all positive potential fields, especially for resting potential, indicating the cancer cells are riot in a normal “resting potential” stage, i.e, about −70 mV, have critically impacted the ratio of action/resting potential (RAPRP), hence the results of RAPRP are larger than that of without incubation. The action potential signals were increased as current increased drastically than that of without incubation. For without incubation, there were superimposed curves for resting potentials regardless the current changes. Current changes had smaller impact on the potentials for without incubation than that of with 24 hrs incubation.

### EXAMPLE 6

### Effect of Current Change on a Normal Breast Cell

Current change effect on a living normal breast cell was illustrated in FIG. 7A. The amplitude of curves at the action and resting potential fields are symmetric alone the zero line, indicating the RAPRP values are close to 0.75-0.9 range, which is at a normal electrophysiological situation. [6]. The sensor is attachable on to the live human breast skin noninvasively. This sensor demonstrated its capability to selectively induce a bio communication more favorably to cancer cell rather than to a normal cell at very sensitive concentration level, because cancer cells with high negative charge density tend to direct hydrogen bonding to the positive imidazolium receptor in the sensor membrane.

### EXAMPLE 7

### The Ratio of “Action Potential/Resting Potential” of Cell Membrane as a Biomarker

The discharge potential was defined as “action potential”, and the charge potential defined as “resting potential. The duration time is 2 s for action or resting potential for the model cancer sensor study. The absolute value of action potential divided by the resting potential was defined as the ratio of action potential vs. resting potential. The ratio was used for assess of cell heat release by a Contour Map Multiple Variable Correlation method (CMMVC).

### EXAMPLE 8

### Assessing Cell Heat Release

The CMMVC method was used for assess of cancer cell heat release. Two variables chosen for assessing the heat released by cancer cells (as Z axis) were 1. Ratio of “Action potential” vs. “Resting potential” (as Y axis) and 2. Cell concentration as X axis was used for cell concentration factor study. Similarly, it was only a change in X axis to current, while other factors are remain the same, was conducted for the current factor study. The results of absolute difference between action and resting potential at a given cell concentration under a known current, were used to multiply the current and then multiply the time duration of the potential fired by the equation of J=I.ΔV.t, I is current in ampere, ΔV is voltage difference in volt and t is time in second. J is Joule. Joule divided by a 4.184 conversion factor gives the calorie released.

### EXAMPLE 9

### Breast Cancer Cell Heat Release Visual Map

The results shown in red hot color in FIGS. 5B and 6B are for the CMMVC visual map for with or without incubation under 5 cancer cell/mL and ±10 mA current conditions. The gradient of red color change was positively correlated with the high abnormal RAPRP as discharge current rose to mA, level for cancer cells and reflected in the visual map. The x axis is log current; the y axis is the ratio value and the z axis is the calorie. After a converting step, the blue color CMMVC map in FIG. 7B illustrates the normal breast cell heat release to the body, which is negligible. The order of magnitude higher in the RAPRP ratio associated with more heat release is the land marker behavior of the breast cancer cells under higher current is demonstrated. In contrast, the normal breast has the RAPRP ratio close to the normal ratio range of 0.75-0.9 with no extra heat was released to the body regardless the current change over 50 pA to 20 mA range.

### EXAMPLE 10

### A Bioelectronic Switcher at the Origin

No one expects a single breast cancer cells can behave like an idea electronic semiconductor switcher and it switches current in opposite flow direction with the switch point at origin as shown in FIG. 9A. The switch “On” and “Off” are at the two cross-points [point 1(−0.0047V, 0.0041A), point 2 (0.005V, −0.0035A) that passes origin (0,0)] about 0.45 ms and 0.8 ms, respectively, are observed. It was the sensor membrane's nanopore structure and the electron-relay function attracted the single breast cancer cell's attention as shown in the art model in FIG. 9B. The cancer cell has magnified the current at 46,074-fold compared with the control that was without cancer cell, indicating the energy of the cancer cell obtained may not only come from stealing the communication active receptor sites for glucose (we did not see the glucose peak, originally it was designed the sensor for detecting glucose [9]), but also from the “Idea Diode” like capability of the cancer cell, in order to maintain its high action membrane potential—a self-compensation of energy system exists, will put cancer cell in an advantage position (Driver seat) than the normal cell. The “Butterfly” characteristic semiconductor I-V curves with high current in ±25 mA when the switch is at “on” position and a very narrow switch window (±5 mV) and short switch time of when the switch is “Off” in less than 1 ms has proven the triple-negative breast cancer's uniqueness in its eternal cell structure, like a “biosemiconductor”.

### EXAMPLE 11

### Evaluation of the Potential Therapeutic Applications

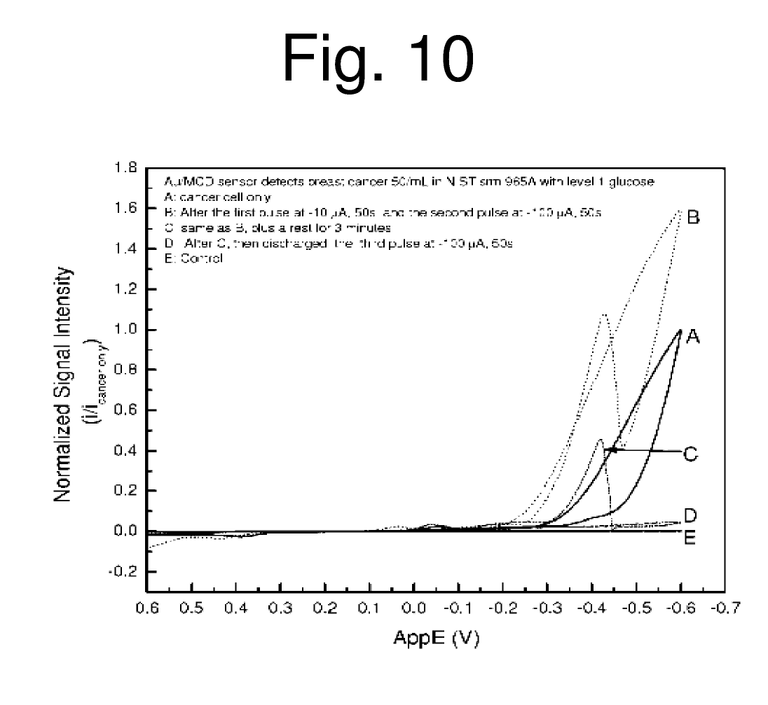

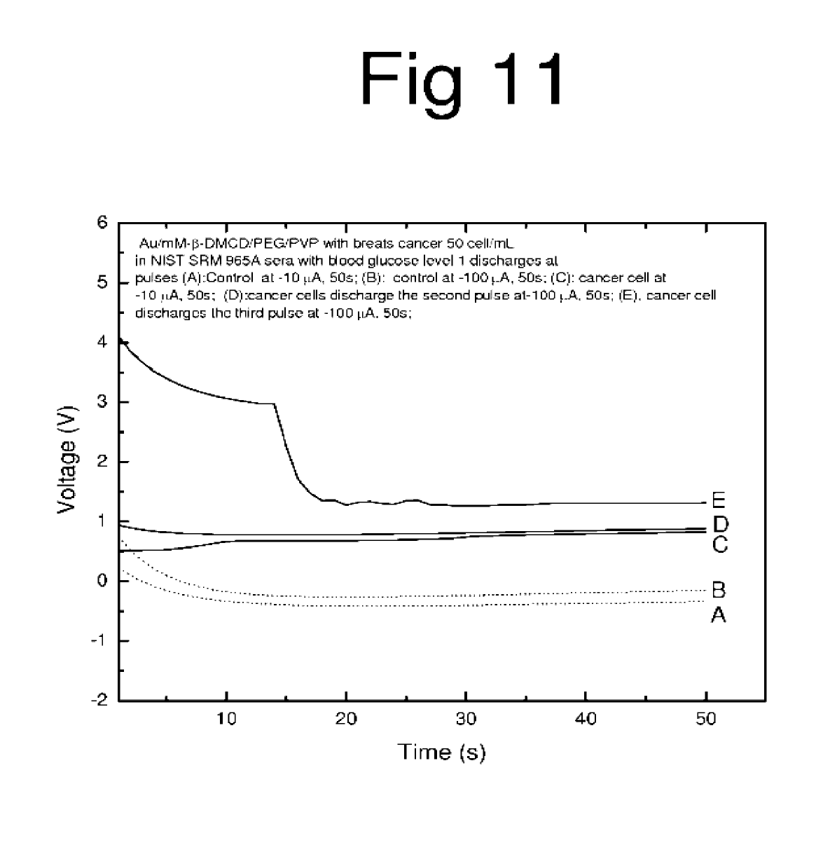

The cancer cells possessed extraordinary high energy than that of normal cell led one to believe that a method to release the extra energy from the cancer cell would be the most effective way to heal cancer than use drugs and radiation. FIG. 10 illustrates CV profiles of pulse effects on signal of live breast cancer cell with the MCD sensor at 50 cell/mL in NIST standard human serum SRM 965A with certified level 1 glucose at room temperature. It was observed that after the first pulse at −10 μA, 50 s as shown in FIG. 11 (C), the peak current intensity at −0.53V suddenly increased by 60% and the appearance of second peak located at −0.33V toward a more positive potential field, along with the cross-points occurred at −0.44V and −0.36V as shown in FIG. 10 (B), that indicate cancer cells urgent to use the “biosemiconducor energy switch” approach to compensate its energy lose, and trying to maintain the membrane active potential as before, hence we observe two switch points along with the signature butterfly curves. There is no butterfly CV curve observed after the second pulse released as shown in FIG. 11 (D) and the peat intensity of the CV curve was reduced by 60% compared with the original peak intensity of cancer alone in FIG. 10 (C), indicates the cancer cell lost its strength and is no longer to maintain the high action potential, that became less aggressive compared with the original peak. After the third pulse as shown in FIG. 11 (E), the cancer peak was totally gone as shown in FIG. 10 (D) against control (E). The benefits of this method are its noninvasiveness pain-free and effective within 3-4 minutes, each pulse lasts 50 s with minimum discomfort, and no side effect. The specific capacitance values are 4.14 mF/g, 40.94 mF/g and 8.20 MF/g at the first 15 s discharge at first, second and third pulses respectively.

The cancer cells enhanced the heterogonous electron transfer rate constant k, 1.54-fold (142/s) compared without the presence of cancer cells in a cell culture media, that the kₛ of the direct electron transfer rate constant is 92/s [11]. The triple-negative breast cancer cell is capable to form an electron-relay network with imidazolium-ATP of cancer cell-water-pyridine at the active sites that mimic the electron-relaying between His 516 and N(5)-FAD of GOₓ as shown in FIGS. 9A and 9B. There was no glucose peak in the curve indicates cancer cell sealed the position for itself even though originally the sensor was designed for glucose [9]. The uniqueness of the sensor here reported is the nanopore structure and the electron-relay network promoted the effective energy transfer between ATP and ADP within the cancer cell as seen the two larger peaks in FIG. 9A, even though the calculation of Ks=αnFν_{c}/RT, where α is the electron transfer coefficient, n is the number of electrons, F is the Faraday constant, ν_{c} is the scan rate, R is the gas constant, and T is temperature [12], is still based on irreversible situation because of the two cross-points exist between the two peaks. This may provide an evidence that the successful energy exchange has accomplished by the “idea semiconductor switchers” of the cancer cells and plus the eternal electron-relay and nanopore structure of the sensor membrane, made the cancer cell regain a momentum.

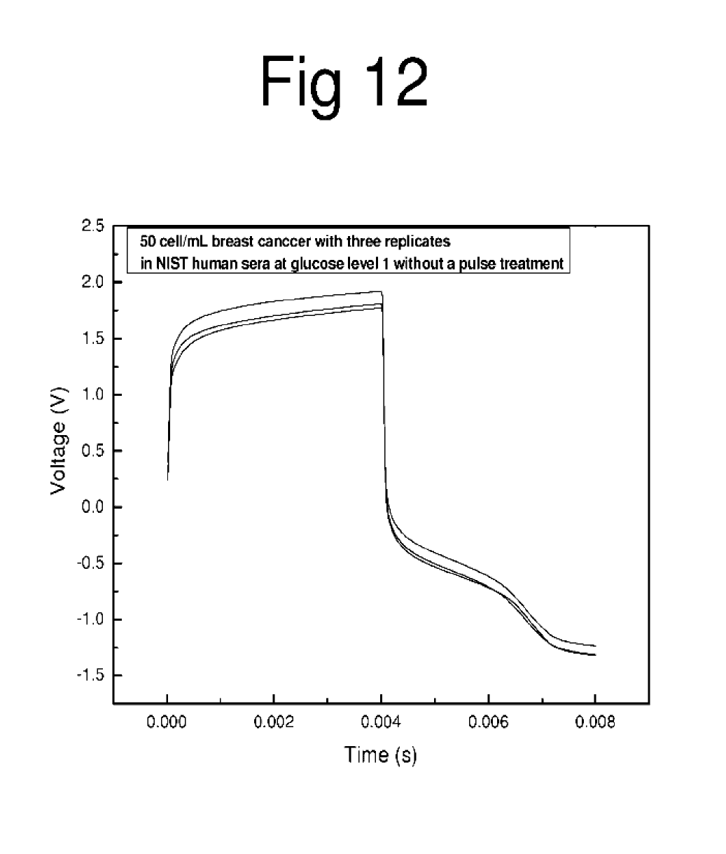

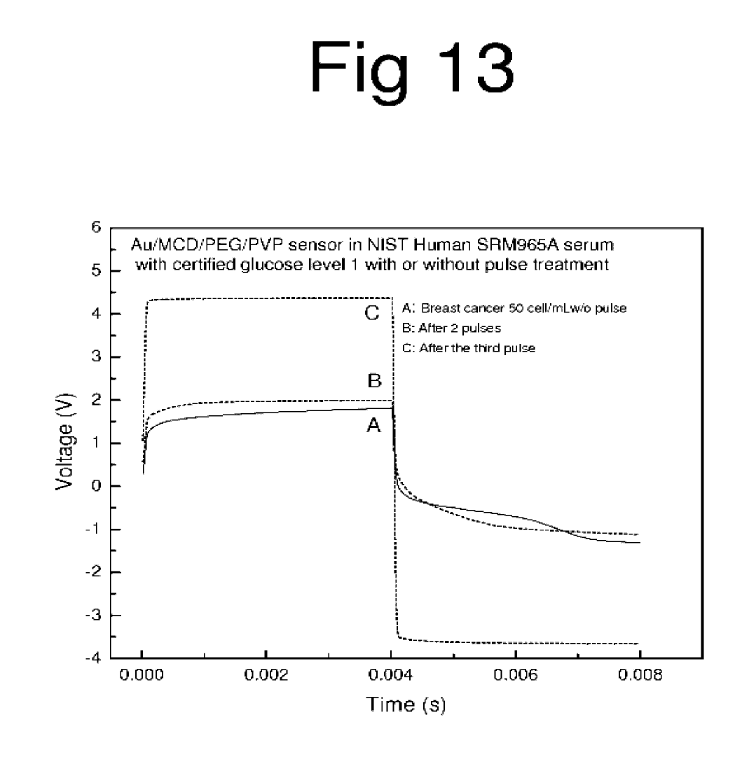

The ratio of the action/resting potential has changes from original 3.2±0.4 (cancer only) as shown in FIGS. 12 to 2.37 and 1.19±0.01, for after second and third pulses, respectively as shown in FIG. 13 (B) and (C) compared with (A) without pulse. The method can restore the asymmetric curves of cancer cell “action/resting potential” to a normal more symmetric curve, and the ratio close to normal cell value of 0.8-0.9, that is close to unity. The CV curves shown in FIG. 10 in D, the cancer finger print signal has completely gone, and reflected as the membrane potential ratio turned to in normal range.

### EXAMPLE 12

### Applications

This contour map of multiple variable correlation method assesses the heat released by cancer cells and monitoring the cancer progress using the nanopore structured Biomimetic device opened a wide areas of applications in all cancer detection areas for visualization of the progress and easy to understand for patients and doctors with 1) higher sensitivity to 1 cell at early stage of cancer vs. late stage of at least 10,000 cancer calls. It can be seen on the image by the mammogram method; 2). Faster in ms to seconds vs. several weeks that the results can be known to patients is beneficial; 3). Higher specificity of only recognizes the single breast cancer cell against other type of cancer cell, like brain cancer cell has no interference with the results against the mammogram method, that can not distinguish brain cancer and breast cancer if both are in the brain; 4.) Portable and small size vs. a big machine for mammogram is benefit; 5) affordability. The new biomarker of ratio of the “Action/Resting” cell membrane potential provides a simplicity parameter for monitoring the cancer progress becomes the key component of the visual map will not only provide accurate, fast, sensitive testing results, but also portable and visual.

### Followings are the Specifications in CIP Application

### EXAMPLE 13

### A Contour Map Display for In vitro Cancer Cells Heat Release after Therapeutic Current Treatments

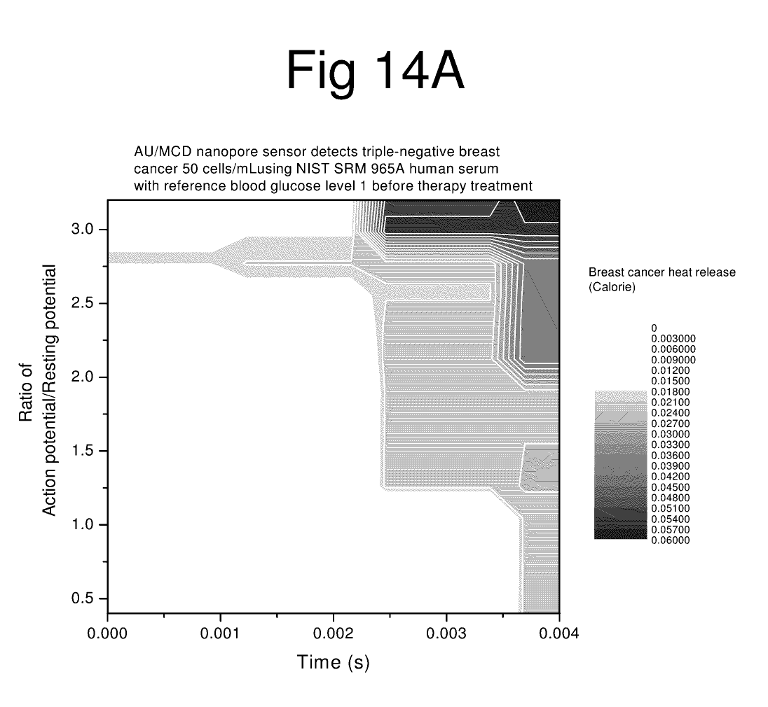

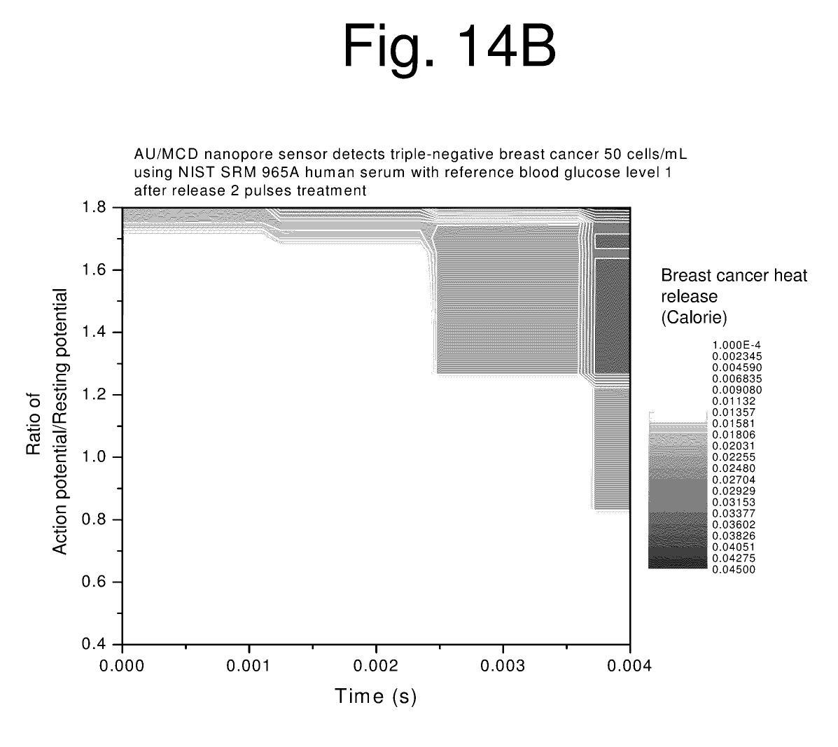

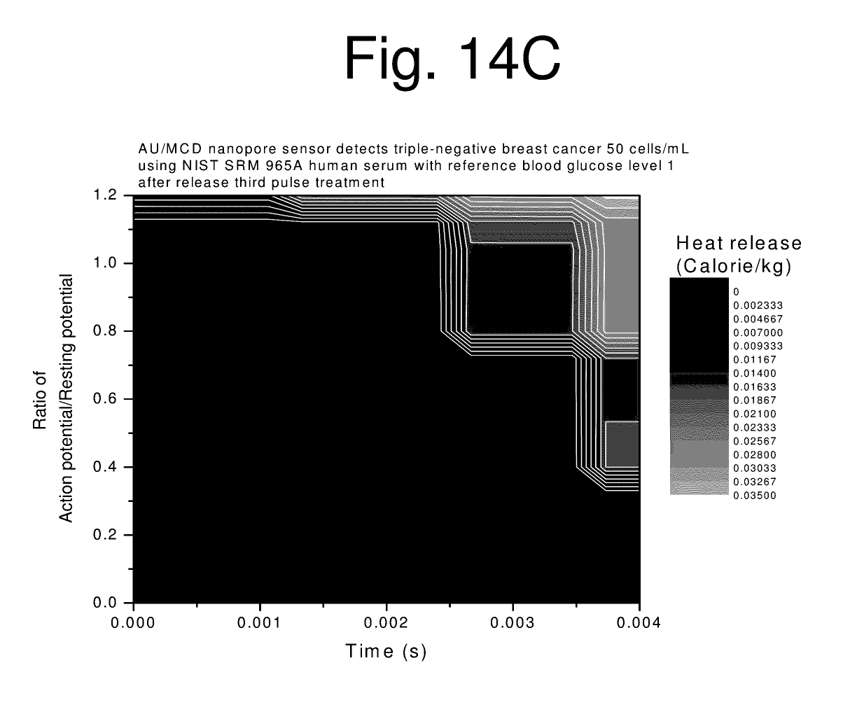

The contour map displays results used for evaluations of in vitro cancer cells heat release after therapeutic current treatments in FIG. 11 was based on the DSCPO evaluation method shown in FIG. 13 were followed up in FIG. 14A, FIG. 14B and FIG. 14C for the cases of before therapeutic treatment, after 2 pulses treatment and after third pulse treatment, respectively. Ratios of action/resting potential reduced from 3.4, 1.8 to 1.2 after pulse treatments, indicating the trend turns to normal. The maps show TNA cancer had no longer release heat after three pulse treatments as seen in the maps.

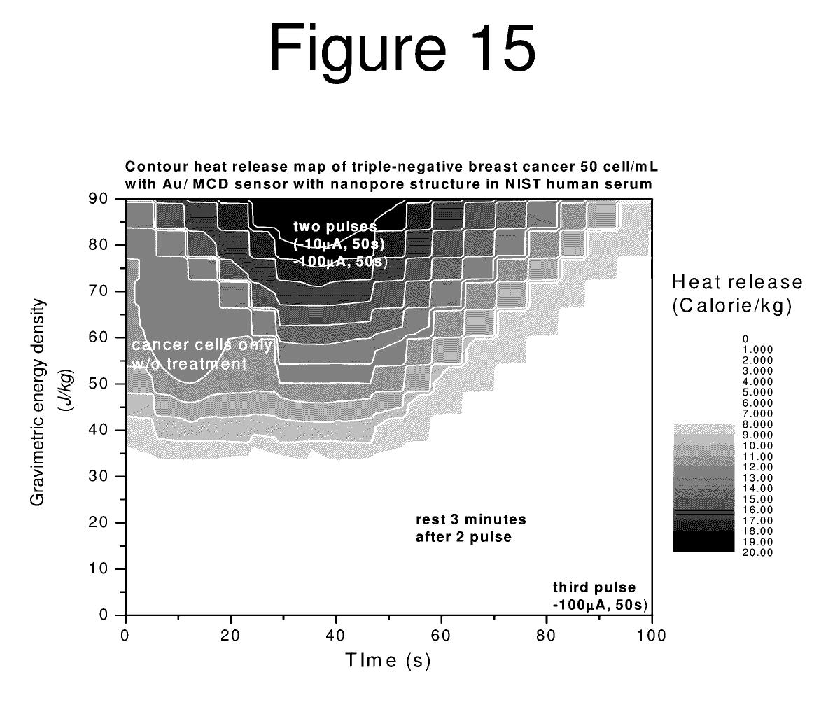

Another contour map displays results used for evaluations of in vitro cancer cells heat release after therapeutic current treatments in FIG. 11 was based on the CV evaluation method shown in FIG. 10 were followed up in FIG. 15, that indicates this method reduced TNA cancer 50 cell/mL strength by 100% after the treatments as shown in FIG. 15 heat release map.

### EXAMPLE 14

### A Method of Applying a Therapeutic Stimulation to Release Heat from Cancer Cells In Vivo

In the Example 9, FIGS. 5B and 6B shown breast cancer cells possessed huge amount of energy and heat. Example 14 describes a therapeutic pulse treatment method to facilitating cancer cells safe release heat by a spontaneous galvanic single step chronopotentiometry method (SSCPO). The method. comprises eight steps of (1) insert a sensor chip with the nanostructured biomimetic membrane described in the Example 16 and Example 17 into the slot of the sensor compartment of the device; (2) fixing the sensor in place at the open window; (3) handheld the device handle and press the exposed sensor surface through the open window onto a cleansed area of the breast; (4) apply a fixed current in μA-mA range according to the individual subject's breast situation started at lower dose at a fixed time interval in seconds to minutes onto the cancer cells in vitro, or onto the breast in vivo outside the areola area and near the duct area; (5) watch the display map color change and movement; (6) rest the breast for 10-20 minutes; (7) apply for second dose if necessary according to the specific situation; (8) repeat the procedure until no longer see the red heat in the map.

### EXAMPLE 15

### Establish a Baseline of In vivo Normal Breast Monitoring Using Heat Release Map

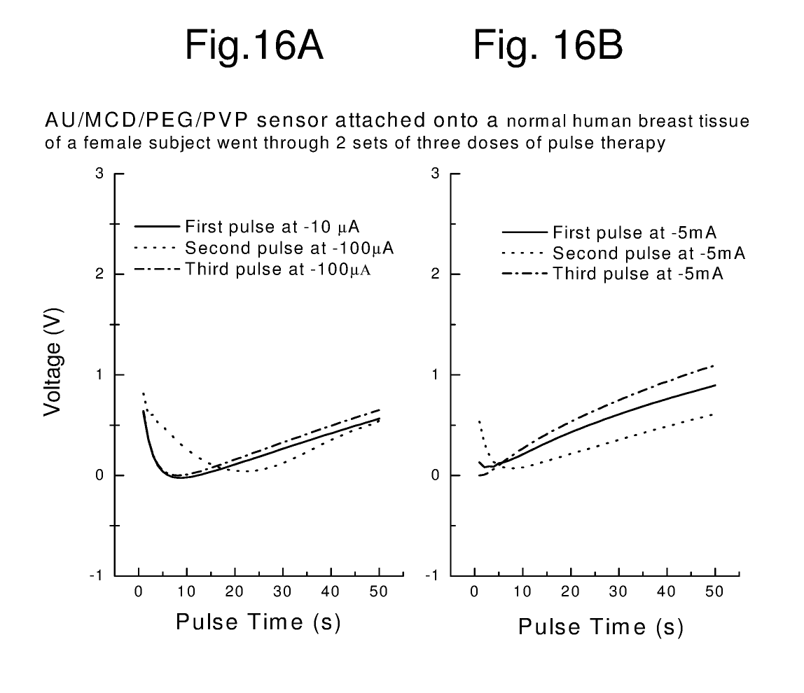

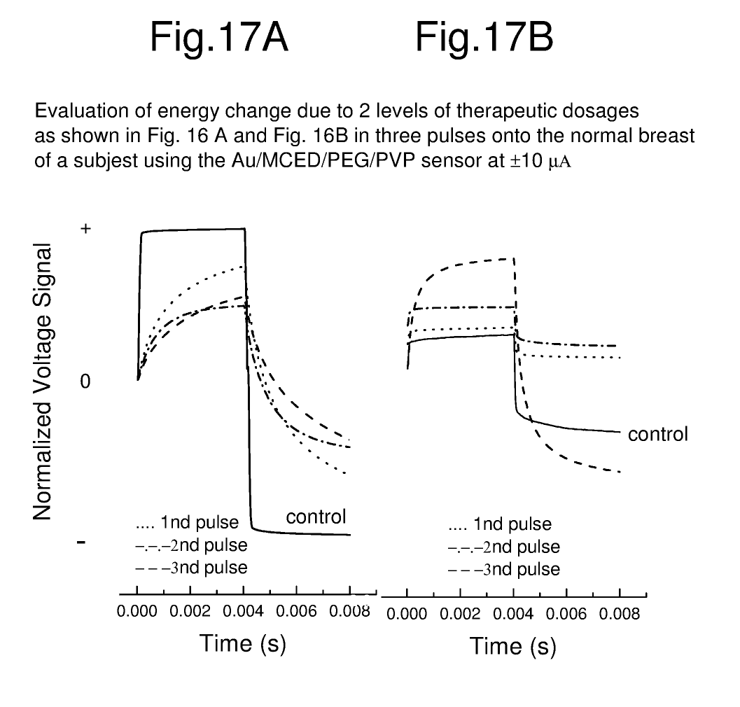

Noninvasive timely monitoring early breast cancer existence is important to promote human health. First FIG. 16A and FIG. 16B present the establishments of baselines of in vivo normal breast monitoring using the SSCPO method. FIG. 16A was for lower dosage in 10-100 μA compared with FIG. 16B in 5 mA at same 50 s pulse duration. It was noticed that the different dosage current applied did not produce significant difference among three pulses of energy discharges on a normal healthy breast in vivo with average voltage outcomes in less than a half volt compared in vitro for cancer cells in FIG. 11, that the third pulse released 3 volt at the first 15 s at s-s state, it is indicating this method has a potential work for patients. The control baseline is repeatable.

Evaluation of energy change at different therapeutic dosages was conducted by a DSCPO method after each pulses shown in FIG. 16A and FIG. 16B using SSCPO method. FIG. 17A depicts the therapeutic dosages as shown in FIG. 16A of −10 μA, −100 μA compared with the control. FIG. 17B depicts the evaluation of energy change as shown in FIG. 16B in −5 mA compared with the controls. We found the normalized figures demonstrate a similar trend that three therapeutic pulses at different levels did not produce significant outcomes on a normal healthy human breast under same evaluation parameters of ±−10 μA and 4 ms. It is noticed the control curves are perfect in biphase symmetric before the therapy with ratio of action potential/resting potential close to unity, and the ratio values in pulses after three pulses therapy are 1.27±0.14 and 1.82±0.31 for lower dose and −5 mA higher dose, respectively. Comparing ratios of before and after cancer treatment in vitro therapy are 3.4 (without treatment), 1.8 (after 2 pulse treatment) and 1.2 (after third pulse treatment), that indicates our lower dose in vivo treatment is safe and the higher dose is at the border. Dosages should not be higher than 5 mA in vivo if one needs go through therapeutic treatment for asymptomatic early cancer monitoring.

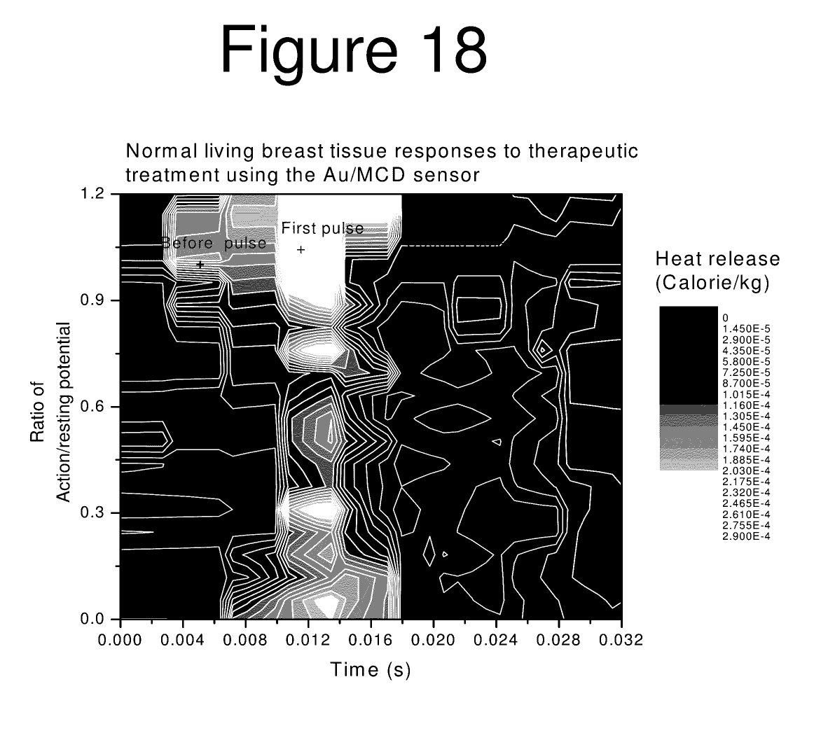

FIG. 18 depicts the heat contour map for monitoring before and after pulse treatment of the energy change as shown in FIG. 17A, the ratios of action/resting potential as the y axis with the values kept around normal range during three pulses treatments compared with before treatment, the x axis values are the total time elapse during the entire treatment, and the z is in heat release calorie/kg. The kg refers to the weight of the sensor. The red labels are for the locations of the pulses. This is a showcase of the low dose treatment for a normal breast for three pulses is safe without red heat release from the map.

### EXAMPLE 15

### Advantages of the Cancer Monitoring and Therapeutic Device System

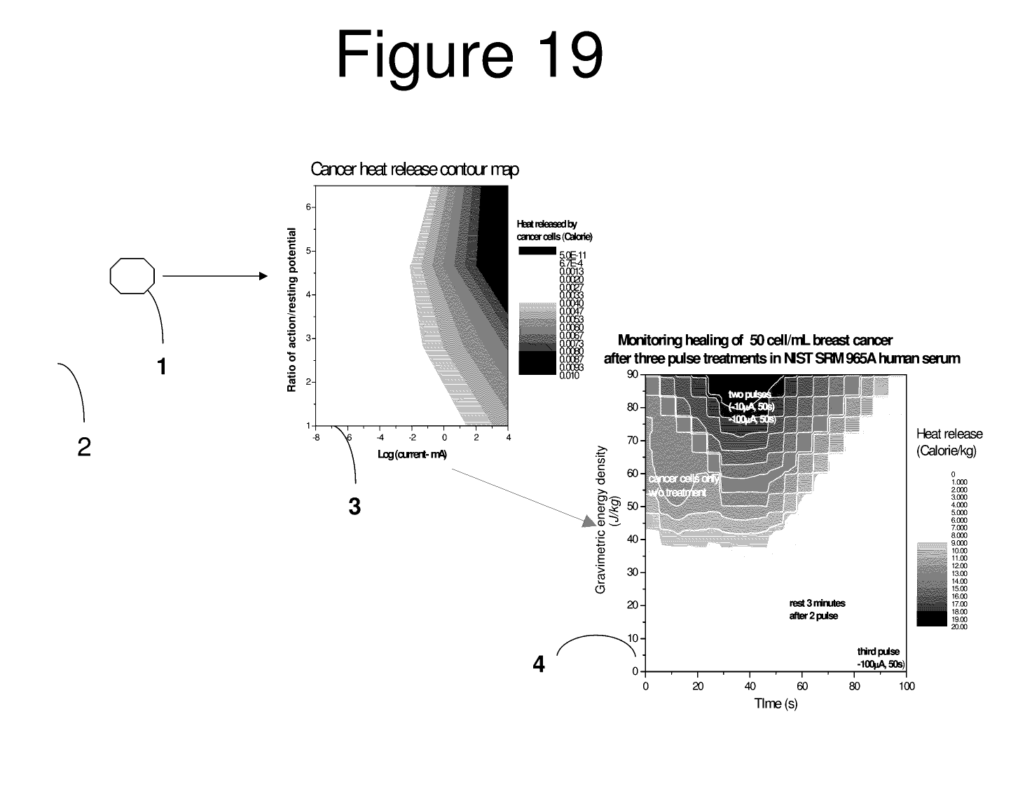

The schematic components of a cancer monitoring and therapeutic device system presented as shown in FIG. 19 comprise of “1” a flexible adhesive sensor pad which will be placed against the skin of the breast “2” and a transmitter with embedded software to transmit the signal wirelessly to a receiver with a receiver/displayer for 3D mapping of heat release in either by ratio of action/resting potential (as y axis), log current as x axis and heat release calorie as Z axis) “3” or by a progress mapping of ratio of action/resting potential as y axis, total pulse therapy time as x axis and heat release calorie as z axis shown in “4”. The sensor/transmitter with embedded software allows a spontaneous electric pulse of potential change at real time is monitored. The current applied is very low in μA and mA range for in vitro and in vivo, respectively and the time duration per dosage therapy is 50 s. Our pulse voltage density for in vitro cancer treatment is 96.8 V/cm² (having the power density 9.6 mW/cm²) compared with the reported article of 238.1 W/cm² based on the voltage density 30 kV/cm having 1 mA current and the electrode area 0.126 cm² [13]; our in vivo human early asymptomatic breast baseline therapeutic record setting for a normal breast tissue treatment is 16.1 V/cm² with the power density of 80 mW/cm² having 5 mA current or 1.6 mW/cm² having 100 μA current. This invention offered no heat release and no scar occurred, in contrast, the reported article had skin scar occurred for the pulse treated mice. The advantage of this invention is not based on an extreme high power density, but rather on the unique nanostructured biomimetic sensor that induced a breast cancer cell selectively communicates with the function groups of the sensor membrane leading to drain the energy causing apoptosis in cancer cell death. The female subject feels no pain in her breast during the treatment and no invasive surgical steps or chemotherapeutic drugs are used and it was totally reagent-free. The 4D contour heat map monitor is displayed on a hand held receiver and shows the color change with time and the ratio change of the action/resting potential with time. The color change in the map represents the viability of the cancer cells being monitored.

### EXAMPLE 16

### Engineering a Prototype Device For Asymptomatic Early Cancer Monitoring and Therapeutic Function

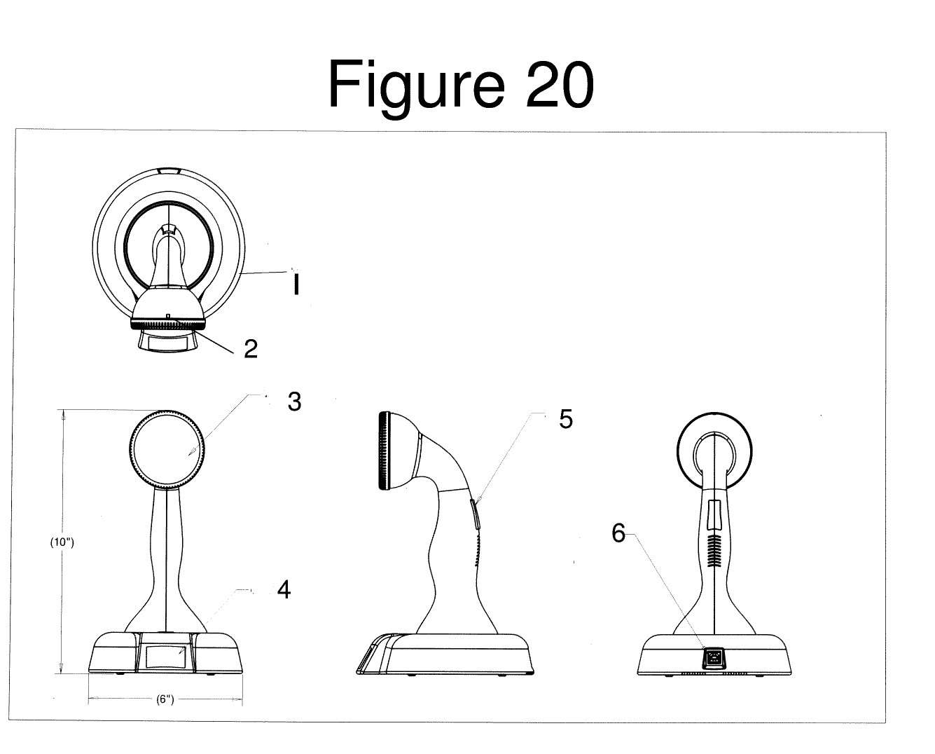

The dynamic cancer monitoring and therapeutic device consists of four parts: 1) a sensor compartment which can be pressed onto the skin of the breast; 2) a transmitter with embedded software of algorism is used for sensor function and data acquisition, A/D signal converter chip to transmit the signal to a receiver; 3) a receiver/displayer with wireless function; 4) a middle part is for hand handle. The sensor/transmitter with embedded software allows a spontaneous electric pulse of potential change at real time is monitored. The designs of a handheld innovative pain-free cancer monitoring and therapeutic device were depicted in FIG. 20 under reagent-free, pain-free and non-invasive conditions. The top figure is a bird-eye view of the device system. “1” represents the bottom base of the device. “2” represents the slid for depositing a sensor. The left side bottom figure is a face-to-face view of the device. It consists of four major parts: the head part is for signal accusation, transmission and conversion; the middle part is for hand grub; the bottom part is for results map display and monitoring and the back has a compartment for USB and wireless interfaces. “3” refers to the contact surface on the head. “4” is the display window for the color coded contour heat release map on the bottom; the middle figure is the side-view of the device. “5” is an activation button. The far right side figure is a back-view of the device with “6” is a USB and wireless interfaces.

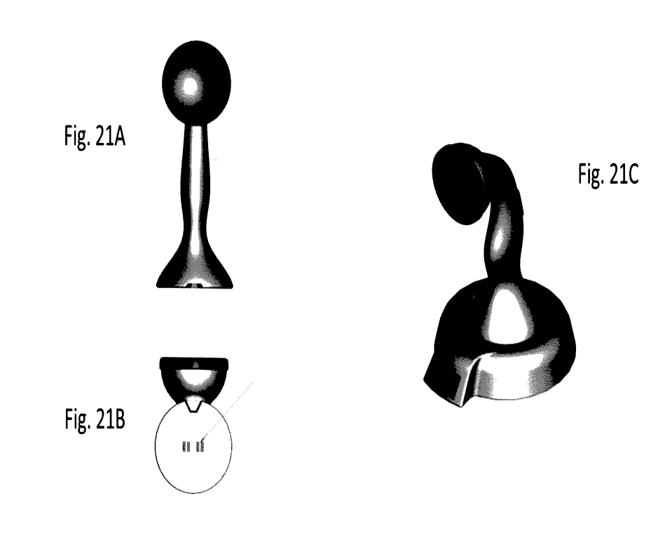

FIG. 21A depicts the face-to-face view of the visual model device. FIG. 21B depicts the bird-view battery charging contacts at the bottom. FIG. 21C is a close view of the visual device with the cancer heat releasing map on the display at the bottom.

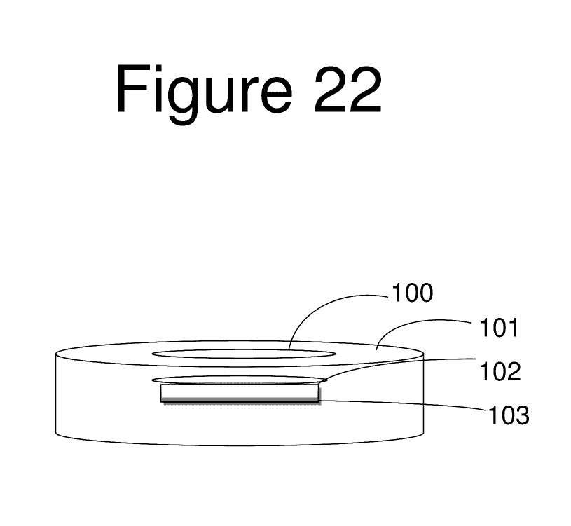

FIG. 22 depicts the components in the top head of the device. “100” is an open window exposing the sensor allowing directly contacts with list, and the sensor was inserted inside the device through a slid shown in “2” of FIG. 20. In FIG. 22, “101” is the device contact surface. “102” is the sensor. “103” is the A/D converter and the embedded software chip for transmission and converting signal and an interface card.

### REFERENCES

1. B. Weiss, G. Ganepola, H. Freeman, Y. Hsu and M. Faupel, Breast Disease, 7(2), 91, (1994).
2. S. V. Sree, E. Ng, G. Kaw, R. Acharya-U, and B. Chong, J. of Medical Systems, 35(1). 79, 2011.
3. A. M. Hassan and M. Ei-Shenawee, Review of electyromagnetic techniques for breast cancer detection, IEEE Transactions in magnetic, on line publisher, 2011.
4. A. M. Hassan and M. Ei-Shenawee, The Diffusion-Drift Algorithm for Modeling the Biopotential Signals of Breast Tumors, #TTT-21, Cancer Detection and Diagnostics Technologies for Global Health Symposium at National Cancer Institute, Aug. 22-23, 2011.
5. D. Lewitus, R. J. Vogelstein, G. Zhen, Y-S Choi, J. Kohn, S. Hershberger, X-F Jia, IEEE Transactions on neuronal systems and rehabilitation engineering 19(2), 204, 2011.
6. L. G. Mitchell, N. A. Campbell, J. B. Reece and M. R. Taylor, Biology, 8^{th} edition, 2007.
7. S. L. Bonifacio, H. C. Glass, D. M. Ferriero and S. Peloquin, Nature Reviews Neruology 7, 485, 2011.
8. E. Chen and H. L. Pardue, Anal Chem. 65, 2563-2567, 1993.
9. E. Chen, Novel Nanopore Structured Electrochemical Biosensor, U.S. Pat. No. 8,083,926, Dec. 27, 2011.
10. E. T. Chen, S-H Duh, C. Ngatchou, J. T. Thornton and P. T. Kissinger, Nanotech (3), 101-104, 2011.
11. E. T. Chen, J. Thornton, C. Ngatchou, S-H. Duh, P. T. Kissinger, study of the correlations between direct electron transfer rate constants and the effectiveness of cancer inhibitors using nanobiomimetic sensors, NSTi-Nanotech 3, 115-118, 2013.
12. E. Laviron, Electroanal Chem., 101, 19-28, 1979.
13. R. Nuccitelli, K. Tran, S. Sheikh et. al., International Journal of Cancer, 127, 1727-1736, 2010.

What is claimed is:

1. A handheld cancer monitoring and therapeutic device comprising:
a sensor compartment comprising an electrode comprising a substrate of gold;
a nanostructured biomimetic self-assembling membrane comprising a polymer matrix comprised of an electrically conductive copolymer comprised of array of nanopores; the nanopores are vertically oriented on the substrate, and wherein the device promotes an electron-relay network with imidazolium-ATP of single cancer cell-water-pyridine at its active sites of the membrane that mimic the electron-relaying between His 516 and N(5)-FAD of GOₓ in the presence of glucose; a transmitter with embedded software of algorism for the sensor function of inducing cancer selective bio-communication;
an A/D signal converter chip; a wireless receiver and displayer and a handle.

2. The device according to claim 1, wherein the copolymer is further comprised of: one or more first β-cyclodextrin molecules having at least one or more imidazolium groups.

3. The device according to claim 1, wherein the membrane comprises with cross-linked polymer.

4. The device according to claim 1, wherein the sensor compartment further comprises an opening window in the center of the sensor compartment, where exposes the electrode to be contacted onto the breast.

5. The device according to claim 4, wherein the open window is perpendicularly orientated to a sensor inserting slot.

6. The device according to claim 5, wherein the sensor inserting slot has a socket for fixing the sensor in the opening window.

7. The use of a device according to claim 1, further comprises steps of insert a sensor chip into the slot of the sensor compartment of the device; fasting the sensor in place at the open window; handheld the device handle and press the exposed sensor surface through the open window contacting with a cleansed area of the breast; apply a fixed current and a fixed time interval in the device, which contacts the testing sample in vitro, or in vivo; watch the display window of results; resting the breast between each dosage therapy treatment; repeat the procedure until no longer see the heat released in the contour color map.

8. The use of a device according to claim 7, the Contour Map based on Multiple Variable Correlation matrix displaying and accessing or detecting cancer cell heat release.

9. The use of a device according to claim 8, wherein the map is a color coded visual map.

10. The use of a device according to claim 9, wherein the mapping method comprises a quantitative map comprised of multiple variables adopted for indications of action/resting potential, current, calorie release, or cancer cell concentration change.

11. The use of a device according to claim 10, wherein the ratio of “Action Potential/Resting Potential” is a special biomarker method, the method comprising use of a special biomarker of the ratio of “Action Potential/Resting Potential” (RAPRP) of cancer cell membrane to monitor cancer progress relative to a normal cell.

12. The use of a device according to claim 11, wherein said the normal values of the RAPRP for a healthy human breast in vivo monitored are in the range between 0.75 to unity over a wide current range applied from ±50 pA to ±20 mA without therapeutic treatment.

13. The device according to claim 1, wherein the device performance in voltage density with in vitro therapeutic treatment of 50 cell/mL TNA cancer cells is 96.8 V/cm² having a power density 9.6 mW/cm².

14. The device according to claim 1, wherein the device performance in voltage density in vivo human early asymptomatic breast baseline therapeutic record setting for a normal breast tissue therapeutic treatment is 16.1 V/cm² with the power density of 80 mW/cm² at a fixed 5 mA current or 1.6 mW/cm² at a fixed 100 μA current.

15. The use of a device according to claim 7, wherein it utilizes 50 cell/mL cancer cell's selective bio-communication with the functional groups of the device membrane inducing spontaneous discharge pulses at fixed current in 10-100 μA range in vitro for three dosages and the cancer possessed extra energy was 100% released.

16. The device according to claim 14, wherein positive outcomes of the therapeutic device transform a cancerous breast cell to normal status through converting an asymmetric voltage discharge curve to a biphasic symmetric discharge curve while a normal range RAPRP value has reached without using radioactive surgery, and no chemotherapy.

17. The device according to claim 1, wherein the therapeutic device is a non-invasive device.

18. The device according to claim 1, wherein the device is free from antibody.

19. The device according to claim 1, wherein the device is free from natural enzymes.

20. The device according to claim 1, wherein the device is no harm to normal breast tissue in vivo therapy under current range less than or equal to 5 mA at 50 kHz.
